(19) United States
(12) Patent Application Publication
(10) Pub. No.: US 2006/0280723 A1
(43) Pub. Date: Dec. 14, 2006

(54) Interferon for treating or preventing a coronaviral infection

(75) Inventor: Karen Elizabeth Jervis, (US)

Correspondence Address: DRINKER BIDDLE & REATH;ATTN: INTELLECTUAL PROPERTY GROUP, ONE LOGAN SQUARE, 18TH AND CHERRY STREETS, PHILADELPHIA, PA, 19103-6996, US

(73) Assignee: Viragen, Inc, Plantation, FL (US)

(21) Appl. No.: 10/557,761

(22) PCT Filed: May 19, 2004
(86) PCT No.: PCT/GB04/02183

§ 371 Date: Nov. 18, 2005

Related U.S. Application Data
(60) Provisional application No. 60/549,575, filed on Mar. 3, 2004.

(30) Foreign Application Priority Data
May 19, 2003 (GB) 0311451.9
Aug. 27, 2003 (GB) 0320018.5

(51) Int. Cl. C12Q 1/70; A61K 38/21

(52) U.S. Cl. 424/85.6; 424/85.7; 435/5

(57) ABSTRACT

The present invention provides a composition and method for use in the prevention or treatment of a coronaviral infection and in particular, the human coronavirus infection termed severe acute respiratory syndrome (SARS) coronavirus (SARS-HCoV). A method of treating a coronaviral infection is provided through the administration of interferon, further the use of interferons in the treatment of a coronaviral infection is also provided. Preferred forms of interferon for use in the invention are multi-subtype interferon products such as multi-subtype, human alpha-interferon derived from white blood cells commercially available as Multiferon.

### FIELD OF THE INVENTION

The present invention provides a composition for use in the treatment or prevention of a coronavirus infection, more specifically a human coronaviral infection, most specifically severe acute respiratory syndrome (SARS) coronavirus.

### BACKGROUND OF THE INVENTION

Viral Infection

Viral infection is initiated by the binding of a viral particle to a receptor on the surface of a host cell membrane. The virus passes into the cell by endocytosis. Enzymes encoded by the viral genome are transcribed by the host cell and cause the viral coat to fuse with the endosome membrane causing the viral genome to be released into the cytosol. The virus uses the host cell to effect protein production in order to make numerous copies of the genome. Viral coats are formed from coat proteins encoded by the viral genome and synthesised by host cell ribosomes. The viral genomes are then packaged into the newly produced viral coats and expelled from the host cell via the intracellular protein trafficking pathway or through cell lysis. The newly synthesised viral particles are then available for infection of other host cells.

Coronaviruses

Members of the order Nidovirales, the coronaviruses are enveloped, single stranded RNA viruses. Coronoviral infection causes severe disease of the respiratory and enteric systems. Coronaviruses have been associated with gastroenteritis, hepatitis, peritonitis and bronchitis. However, infection in humans generally results in milder symptoms. The SARS human coronavirus (SARS-HCoV) appears to be the first coronavirus which regularly causes severe disease in humans. SARS-HCoV causes severe pneumonia-like symptoms in those infected, with mortality occurring in the most severe cases.

Treatment of SARS

Various anti-viral treatments have been administered to humans infected with SARS-HCoV, including general anti-virals, treatments which inhibit viral cell entry or replication, and immunostimulants.

Ribavirin is a broad spectrum anti-viral agent based on a purine nucleoside analogue and is the standard treatment regimen for hepatitis C. Ribavirin is known to be active against various RNA viruses by inducing lethal mutagenesis of the viral RNA genome (Crotty et al., 2000; Tam et al., 2001) and is known to show anti-viral activity against animal coronaviruses (Weiss & Oostrom-Ram, 1989; Sidwell et al., 1987). However, in vitro tests of the efficacy of the drug against SARS-HCoV have produced a series of negative results and adverse reactions have also been reported.

A limited number of other drugs have undergone testing. The influenza drug, Oseltamivir, a neuramidase inhibitor, has undergone analysis for its efficacy against SARS-HCoV infection, but has not shown any therapeutic benefit (Lee et al., 2003 and Poutanen et al., 2003). In laboratory tests, Cystatin C, a protease inhibitor found in human blood, was found to block replication of the ‘common cold’ coronaviruses, but this has not been tested against SARS-HCoV. It is unlikely that Cystatin C will be a candidate for the treatment of SARS-HCoV infected patients, since it has not undergone the safety and efficacy tests required for all human therapeutics.

Interferons

The interferons (IFNs) may be classified into two distinct types—Type I IFNs and the Type II IFNs. The type I IFNs consist of IFN alpha and IFN beta, whereas the Type II group consists of IFN gamma. Type I IFNs are produced in direct response to a viral infection.

IFN alpha is represented by a large family of structurally related genes expressing at least thirteen subtypes, whereas IFN beta is encoded by a single gene (Diaz et al., 1996). Both types of IFN are able to stimulate an anti-viral state in target cells, whereby the replication of a virus is inhibited through the synthesis of enzymes which interfere with the cellular and viral processes.

Type I IFNs also act to inhibit or slow the growth of target cells and may render them more susceptible to apoptosis. This has the effect of limiting the extent of viral spread. Type I IFNs are immunomodulators, or ‘biological response modifiers’ which act to stimulate the immune response. Even though IFN alpha and IFN beta show many broad similarities in their actions, there are significant differences in the manner by which they exert their effects and it is these extended functions that account for the different ranges of antiviral activities of the two types. A review of the different mechanisms by which interferons exert their anti-viral effects is provided by Goodbourn et al., 2000.

Recombinant interferons, which consist of only the IFN alpha 2 subtype, currently dominate the market for anti-viral and oncology indications. The two main recombinant alpha IFN products, Intron A™ from Schering Plough (IFN-alpha 2b) and Roferon™ (IFN-alpha 2a) from Roche. In contrast to these single-subtype products, there are several alpha IFN preparations that consist of a mixture of different subtypes. These multi-subtype IFN alpha products are produced either by human leukocytes in response to a stimulation from a virus (such as Multiferon™ from Viragen, Inc or its subsidiaries, or Alferon-N™ from Interferon Sciences/Hemispherix), or in human lymphoblastoid cells, cultured from a patient with Burkitt's lymphoma (such as Sumiferon™ from Sumitomo).

There are many differences between the recombinant forms of IFN alpha and the multi-subtype forms. The most obvious difference is the number of IFN alpha subtypes each possesses. As mentioned previously, the recombinant forms comprise only the alpha 2 subtype—the alpha 2b form for Intron A™ (Schering Plough) and the alpha 2a form for Roferon™ (Roche). These two allelic variants differ by only one amino acid residue. The multi-subtype forms of IFN alpha, as the name suggests, comprise many subtypes of IFN alpha. Another difference between the multi-subtype and the recombinant forms is that the IFN alpha 2 produced by human cells in the manufacturing process of the multi-subtype forms is glycosylated, whereas the recombinant forms are unglycosylated, in that they are produced through bacterial fermentation. Glycosylation plays a major role in many functions of the protein product, such as half-life, the bioactivity and its immunogenicity. Therefore, the glycosylation of a product is an important consideration when developing a therapeutic or prophylactic treatment, as it may affect the duration in the body after administration, the activity of a therapeutically appropriate dose and the tolerability to the product itself.

During the last decade, considerable progress has been achieved in the identification of the components, as well as the molecular events involved in the immunotherapeutic effects of interferons. Over thirty different proteins have been identified that have been shown to be induced by interferon (Strannegard, 2002, unpublished review).

There are currently no completely effective therapeutic or prophylactic treatments for humans infected with coronavirus and in particular SARS-HCoV. There thus exists a need for an effective treatment for coronaviral infection in humans, and in particular for severe acute respiratory syndrome (SARS) coronavirus.

### SUMMARY OF THE INVENTION

The present inventors have now shown that interferons and in particular multiple subtype natural human alpha interferon products are surprisingly effective at treating human coronavirus infection, and in particular severe acute respiratory syndrome (SARS) coronavirus.

According to a first aspect of the present invention there is provided a method of treating coronaviral infection, the method including the step of administering a therapeutically useful amount of an interferon to a subject in need of treatment.

In one preferred embodiment, the method of treatment can be used to prevent coronaviral infection, the method including the step of administering a therapeutically useful amount of an interferon to a subject sufficient to cause protection against infection.

Interferon in each or any of the aspects of the invention is preferably isolated interferon. An isolated interferon is an interferon which is synthetic (e.g. recombinant), or which is altered, removed or purified from the natural state through human intervention. For example, an interferon naturally present in a living animal is not isolated, whereas a synthetic interferon, or an interferon which is partially or completely separated from the coexisting materials of its natural state, is isolated. An isolated interferon can exist in substantially purified form, or can exist in a non-native environment such as, for example, a cell into which the interferon has been introduced. Interferons purified from human cells, for example the multi-subtype, human alpha-interferon derived from white blood cells commercially available as Multiferon™ from Viragen, Inc. or any of its subsidiaries, are also considered to be isolated molecules for purposes of the present invention.

The interferon may be any suitable interferon, for example interferon alpha or interferon beta. It may be single or multi-subtype, but is preferably multi-subtype.

The interferon may be naturally derived, for example from human cells or recombinant, but preferably the interferon is naturally derived. Preferably the naturally derived interferon is obtained from leukocytes following viral stimulation or produced in human lymphoblastoid cells cultured from a patient with Burkitt's lymphoma.

Preferred interferons for use in the invention include multi-subtype interferon alpha (IFNα), interferon αn1, interferon αn3 or interferon β1b. A particularly preferred interferon for use in the invention is the multi-subtype IFNα product commercially available from Viragen, Inc. or any of its subsidiaries under the trade name Multiferon™.

As used herein the term Multiferon™ refers to a highly purified, multi-subtype, human alpha interferon derived from human white blood cells commercially available from Viragen, Inc or any of its subsidiaries.

According to a second aspect of the present invention there is provided an interferon for use in the treatment or prevention of a coronaviral infection.

Preferably the interferon is an isolated interferon.

The interferon may be any suitable interferon, for example interferon alpha or interferon beta. It may be single or multi-subtype, but is preferably multi-subtype.

The interferon may be naturally derived, for example from human cells or recombinant, but preferably the interferon is naturally derived. Preferably the naturally derived interferon is obtained from leukocytes following viral stimulation or produced in human lymphoblastoid cells cultured from a patient with Burkitt's lymphoma.

Preferred interferons for use in the invention include multi-subtype interferon alpha (IFNα), interferon αn1, interferon αn3 or interferon β1b. A particularly preferred interferon for use in the invention is the multi-subtype IFNα product commercially available from Viragen, Inc. or any of its subsidiaries under the trade name Multiferon™.

As used herein the term Multiferon™ refers to a highly purified, multi-subtype, human alpha interferon derived from human white blood cells commercially available from Viragen, Inc or any of its subsidiaries.

According to a third aspect of the present invention there is provided the use of an interferon in the preparation of a medicament for the treatment or prevention of a coronaviral infection.

Preferably the interferon is an isolated interferon.

The interferon may be any suitable interferon, for example interferon alpha or interferon beta. It may be single or multi-subtype, but is preferably multi-subtype.

The interferon may be naturally derived, for example from human cells or recombinant, but preferably the interferon is naturally derived. Preferably the naturally derived interferon is obtained from leukocytes following viral stimulation or produced in human lymphoblastoid cells cultured from a patient with Burkitt's lymphoma.

Preferred interferons for use in the invention include multi-subtype interferon alpha (IFNα), interferon αn1, interferon αn3 or interferon β1b. A particularly preferred interferon for use in the invention is the multi-subtype IFNα product commercially available from Viragen, Inc. or any of its subsidiaries under the trade name Multiferon As used herein the term Multiferon™ refers to a highly purified, multi-subtype, human alpha interferon derived from human white blood cells commercially available from Viragen, Inc or any of its subsidiaries.

Preferably the coronaviral infection is a human coronaviral infection. Most preferably the coronaviral infection is severe acute respiratory system (SARS) coronavirus (SARS-HCoV).

According to a fourth aspect of the present invention there is provided a method of treating or preventing human infection with a coronavirus, and in particular severe acute respiratory system (SARS) coronavirus (SARS-HCoV), the method including the step of administering a therapeutically useful amount of an interferon to a subject in need of treatment along with a therapeutically useful amount of a suitable anti-viral compound.

In one preferred embodiment, the method of treatment includes the prevention of human infection with a coronavirus, wherein the method includes the step of administering a therapeutically useful amount of an interferon, or administering an amount of an interferon along with an amount of a suitable anti-viral compound sufficient to cause protection against the infection.

Preferably the interferon is an isolated interferon.

Preferably the anti-viral compound is ribavirin.

Preferably the interferon is any suitable interferon, for example interferon alpha or interferon beta. It may be single or multi-subtype, but is preferably multi-subtype.

The interferon may be naturally derived, for example from human cells or of recombinant form, but preferably the interferon is naturally derived. Preferably the naturally derived interferon is obtained from leukocytes following viral stimulation or produced in human lymphoblastoid cells cultured from a patient with Burkitt's lymphoma.

Preferred interferons for use in the invention include multi-subtype interferon alpha (IFNα), interferon αn1, interferon αn3 or interferon β1b. A particularly preferred interferon for use in the invention is the multi-subtype IFNα product commercially available from Viragen, Inc. or any of its subsidiaries under the trade name Multiferon™.

As used herein the term Multiferon™ refers to a highly purified, multi-subtype, human alpha interferon derived from human white blood cells commercially available from Viragen, Inc or any of its subsidiaries.

According to a fifth aspect of the present invention there is provided the use of interferon and an anti-viral compound in the preparation of a combined medicament for the treatment or prevention of infection with a coronavirus, and in particular severe acute respiratory system (SARS) coronavirus (SARS-HCoV).

Preferably the interferon is an isolated interferon.

Preferably the anti-viral compound is ribavirin.

Preferably the interferon is any suitable interferon, for example interferon alpha or interferon beta. It may be single or multi-subtype, but is preferably multi-subtype.

The interferon may be naturally derived, for example from humans cell, or of recombinant form, but preferably the interferon is naturally derived. Preferably the naturally derived interferon is obtained from leukocytes following viral stimulation or produced in human lymphoblastoid cells cultured from a patient with Burkitt's lymphoma.

Preferred interferons for use in the invention include multi-subtype interferon alpha (IFNα), interferon αn1, interferon αn3 or interferon β1b. A particularly preferred interferon for use in the invention is the multi-subtype IFNα product commercially available from Viragen, Inc. or any of its subsidiaries under the trade name Multiferon™.

As used herein the term Multiferon™ refers to a highly purified, multi-subtype, human alpha interferon derived from human white blood cells commercially available from Viragen, Inc or any of its subsidiaries.

The term ‘treatment’ as used herein refers to any regime that can benefit a human or non-human animal. The treatment may be in respect of an existing condition or may be prophylactic (preventative treatment). Treatment may include curative, alleviation or prophylactic effects.

Administration

Interferons of and for use in the present invention may be administered alone, or in combination with another agent, but will preferably be administered as a pharmaceutical composition, which will generally comprise a suitable pharmaceutical excipient, diluent or carrier selected dependent on the intended route of administration.

Interferons of and for use in the present invention may be administered to a patient in need of treatment via any suitable route. The precise dose will depend upon a number of factors, including the precise nature of the interferon.

Some suitable routes of administration include (but are not limited to) oral, rectal, nasal, topical (including buccal and sublingual), vaginal or parenteral (including subcutaneous, intramuscular, intravenous, intradermal, intrathecal and epidural) administration, or administration via oral or nasal inhalation.

In preferred embodiments, the composition is deliverable as an injectable composition, is administered orally, is administered to the lungs as an aerosol via oral or nasal inhalation.

For administration via the oral or nasal inhalation routes, preferably the active ingredient will be in a suitable pharmaceutical formulation and may be delivered using a mechanical form including, but not restricted to an inhaler or nebuliser device.

Further, where the oral or nasal inhalation routes are used, administration by a SPAG (small particulate aerosol generator) may be used.

For intravenous injection, the active ingredient will be in the form of a parenterally acceptable aqueous solution which is pyrogen-free and has suitable pH, isotonicity and stability. Those of relevant skill in the art are well able to prepare suitable solutions using, for example, isotonic vehicles such as Sodium Chloride Injection, Ringer's Injection, Lactated Ringer's Injection. Preservatives, stabilisers, buffers, antioxidants and/or other additives may be included, as required.

Pharmaceutical compositions for oral administration may be in tablet, capsule, powder or liquid form. A tablet may comprise a solid carrier such as gelatin or an adjuvant. Liquid pharmaceutical compositions generally comprise a liquid carrier such as water, petroleum, animal or vegetable oils, mineral oil or synthetic oil. Physiological saline solution, dextrose or other saccharide solution or glycols such as ethylene glycol, propylene glycol or polyethylene glycol may be included.

The composition may also be administered via microspheres, liposomes, other microparticulate delivery systems or sustained release formulations placed in certain tissues including blood. Suitable examples of sustained release carriers include semipermeable polymer matrices in the form of shared articles, e.g. suppositories or microcapsules. Implantable or microcapsular sustained release matrices include polylactides (U.S. Pat. No. 3,773,919 and European Patent Application Publication No 0,058,481) copolymers of L-glutamic acid and gamma ethyl-L-glutamate (Sidman et al., Biopolymers 22(1): 547-556, 1985), poly(2-hydroxyethyl-methacrylate) or ethylene vinyl acetate (Langer et al., J. Biomed. Mater. Res. 15: 167-277, 1981, and Langer, Chem. Tech. 12:98-105, 1982, the entire disclosures of which are herein incorporated by reference).

Examples of the techniques and protocols mentioned above and other techniques and protocols which may be used in accordance with the invention can be found in Remington's Pharmaceutical Sciences, 16th edition, Oslo, A. (ed), 1980, the entire disclosure of which is herein incorporated by reference

Pharmaceutical Compositions

As described above, the present invention extends to a pharmaceutical composition for the treatment or prevention of a coronaviral infection, wherein the composition comprises at least one interferon. Pharmaceutical compositions according to the present invention, and for use in accordance with the present invention may comprise, in addition to active ingredient (i.e. one or more interferons), a pharmaceutically acceptable excipient, carrier, buffer stabiliser or other materials well known to those skilled in the art. Such materials should be non-toxic and should not interfere with the efficacy of the active ingredient. The precise nature of the carrier or other material will depend on the route of administration, which may be, for example, oral, intravenous, or intranasal.

The formulation may be a liquid, for example, a physiologic salt solution containing non-phosphate buffer at pH 6.8 to 7.6, or a lyophilised powder.

Dose

The composition/interferon is preferably administered to an individual in a “therapeutically effective amount”, this being sufficient to show benefit to the individual. The actual amount administered, and rate and time-course of administration, will depend on the nature and severity of what is being treated. Prescription of treatment, e.g. decisions on dosage etc, is ultimately within the responsibility and at the discretion of general practitioners and other medical doctors, and typically takes account of the disorder to be treated, the condition of the individual patient, the site of delivery, the method of administration and other factors known to practitioners.

The optimal dose can be determined by physicians based on a number of parameters including, for example, age, sex, weight, severity of the condition being treated, the active ingredient being administered and the route of administration.

For example, in one embodiment, a suitable dose of interferon may be 1 to 10 million IU, for example 3 to 5 million IU three times weekly to 0.5 to 10 million, for example 2 to 8 million, or 4 to 6 million IU daily, although other doses may be used.

According to a further aspect of the present invention there is provided an assay method for determining the efficacy of a composition in the treatment or prevention of a coronaviral infection, wherein the composition comprises an interferon, preferably a multi sub-type interferon.

In a further aspect of the present invention, there is provided an assay method for determining the efficacy of a candidate agent in the treatment of a coronaviral infection, wherein the assay method includes the steps of;
incubating virus infected cells in the presence of the candidate agent, and
determining the degree of inhibition of the cytopathic effect of the virus on the cells.

Preferably the method includes the further step of comparing the degree of viral inhibition obtained using the candidate agent with the degree of viral inhibition obtainable with incubation with an interferon or interferon based product.

Preferably the interferon is a multi-subtype interferon, most preferably Multiferon™.

In a still further aspect, there is provided an assay method for determining the efficacy of a candidate agent in the prevention of a coronaviral infection, wherein the assay method includes the steps of:

incubating cells in the presence of the candidate agent,

adding the coronavirus to the cells, and

determining the degree of protection against the coronaviral infection afforded by the candidate agent

Preferred assays for use in the assay methods of the invention include cytopathic endpoint assays and plaque reduction assays.

Preferred features of each aspect of the invention are as for each of the other aspects mutatis mutandis unless the context demands otherwise.

Unless otherwise defined, all technical and scientific terms used herein have the meaning commonly understood by a person who is skilled in the art in the field of the present invention

Throughout the specification, unless the context demands otherwise, the terms ‘comprise’ or ‘include’, or variations such as ‘comprises’ or ‘comprising’, ‘includes’ or ‘including’ will be understood to imply the inclusion of a stated integer or group of integers, but not the exclusion of any other integer or group of integers.

### DETAILED DESCRIPTION OF THE INVENTION

The present invention will now be described with reference to the following examples which are provided for the purpose of illustration and are not intended to be construed as being limiting on the present invention, and further, with reference to the figures.

### BRIEF DESCRIPTION OF THE DRAWINGS

FIG. 1 shows a dose response curve produced from an in vitro plaque reduction assay, showing that with increasing concentrations of the Multiferon™, the effect of the SARS-HCoV virus is attenuated;

FIG. 2 shows the effect of Multiferon™ and Intron A™ on the cytopathogenicity of Semliki Forest Virus (SFV) on African Green Monkey Kidney Vero E6 cells;

FIG. 3 shows the effect of Multiferon™ on the cytopathogenicity of human Encephalomyocarditis virus (EMCV) on human A459 cells, wherein the Multiferon™ concentration required to obtain 50% cytopathic effect (CPE) for human A459 cells challenged with EMC virus is shown for different concentrations of EMC virus, presented as a 1/dilution; and

FIG. 4 shows the effect of increasing concentrations of Multiferon™ on human A459 cell survival. Cell survival was measured photometrically at Abs_{595 nm} using a fixed dilution of EMC virus (dilution 1/400), at increasing concentrations of Multiferon. AU denoted Absorbance Units.

### EXAMPLES

### Example 1

### Anti-Viral Effect of Interferon Against SARS-HCoV Infection in Vero E6 Cells

The effectiveness of the interferons to inhibit the cytopathic effect following SARS-HCoV infection was tested in a cytopathic endpoint assay and a plaque reduction assay. All endpoint assays were carried out using the multi-subtype interferons Multiferon™ and interferon αn3, as well as single subtype recombinant interferon alpha (subtypes interferon α2a, interferon α2b, and interferon αn1) and the interferon beta (IFNβ) subtypes interferon β1a and interferon β1b as well as the anti-viral Ribavirin for comparison.

Preparation of Anti-Viral Treatments

A broad range of concentrations (obtained by ten-fold dilutions) encompassing the inhibitory dosages stated by the manufacturer for other viral-host combinations was tested. Compounds already present in aqueous injections were made up to volume using Hank's buffered saline solution. For tablet and capsule formulations with soluble active ingredients, the outer coat was removed wherever applicable and the preparation ground in a mortar and pestle. The contents were dissolved in water, vortexed and centrifuged thereafter at 3000 G. The required volume was pipetted from the supernatant and diluted accordingly. Where active ingredients were insoluble in water, the contents were dissolved in dimethylsulphoxide (DMSO) and care was taken to ensure that the final concentration of DMSO in the dilutions would not exceed 1%. For plaque assays, 5-fold drug dilutions were prepared using growth media as specified below.

SARS-HCoV Production and Infection

African Green Monkey (Vero E6) cells (American Type Culture Collection, Manassas, Va., USA) were propagated in 75 cm² cell culture flasks containing growth medium consisting of medium 199 (Sigma, St Louis, USA) supplemented with 10% foetal calf serum (FCS; Biological Industries, Israel). SARS-HCoV 2003VA2774 (an isolate from a SARS patient in Singapore) was propagated in Vero E6 cells. Briefly, 2 ml of stock virus was added to a confluent monolayer of Vero E6 cells and incubated at 37° C. in 5% CO₂ for one hour. 13 ml of medium 199 supplemented with 5% FCS was then added. The cultures were incubated at 37° C. in 5% CO₂ and the inhibition of cytopathic effect gauged by observing each well through an inverted microscope. Where 75% or greater inhibition was observed after 48 hours, the supernatant was harvested. The supernatant was clarified at 2500 rpm and then aliquoted into cryovials and stored at −80° C. until use.

Virus Handling and Titration

Virus titre in the frozen culture supernatant was determined using a plaque assay carried out in duplicate. Briefly, 100 microlitres of virus in 10-fold serial dilution was added to a monolayer of Vero E6 cells in a 24 well-plate. After incubation for an hour at 37° C. in 5% CO₂, the viral inoculum was aspirated and 1 ml of carboxymethylcellulose overlay with medium 199 supplemented with 5% FCS was added to each well. After four days of incubation, the cells were fixed with 10% formalin and stained with 2% crystal violet. The plaques were counted visually and the virus titre in plaque forming units per ml (pfu/ml) calculated.

Cytopathic Endpoint Assay

The protocol used was adapted from Al-Jabri et al. 1996. The effect of each anti-viral treatment was tested in quadruplicate. Briefly, 100 microlitres of serial 10-fold dilutions of each treatment were incubated with 100 microlitres of Vero E6 cells giving a final cell count of 20,000 cells per well in a 96-well plate. Incubation was at 37° C. in 5% CO₂ overnight for the interferon preparations and for one hour for Ribavirin. 10 microlitres of virus at a concentration of 10,000 pfu/well were then added to each test well. This equates to a multiplicity of infection (MOI) (virus particles per cell) of 0.5. The plates were incubated at 37° C. in 5% CO₂ for three days and the plates were observed daily for cytopathic effects. The end point was the diluted concentration that inhibited the cytopathic effect in all four set-ups (CIA₁₀₀).

To determine cytotoxicity, 100 microlitres of serial 10-fold dilutions of each treatment were incubated with 100 microlitres of Vero E6 cells giving a final cell count of 20,000 cells per well in a 96-well plate, without viral challenge. The plates were then incubated at 37° C. in 5% CO₂ for three days and toxicity effects were observed for using an inverted microscope.

Interferons which showed complete inhibition were tested further at the lower viral titres of 10³ and 10² pfu/well.

Plaque Reduction Assay

Multiferon™, interferon αn3 and interferon β1b were further tested using a plaque reduction assay. Trypsinised Vero E6 cells were re-suspended in growth medium and pre-incubated for 15 hours with a serial 5-fold dilution of interferon αn3, interferon β1a and Multiferon™ in 24-well plates. The following day, the medium was aspirated and 100 microlitres of virus was added to each well at a titre of 100 pfu/well.

After incubation for one hour, the virus inoculum was aspirated and a carboxymethylcellulose overlay containing maintenance medium and the appropriate interferon concentration was added. After four days incubation, the plates were fixed and stained as described above.

Viral plaques were visible 3 days after pre-incubation of infected cells for 15 hours with five-fold dilutions of the interferon. Plaques were then counted visually and the concentration of the interferon which inhibited 50% of plaques in each well (IC₅₀) determined. Results were plotted in Microsoft Excel, and a polynomial of order three was used to approximate the data and extrapolate IC₅₀ and IC₉₅ values. (Results not shown)

The assay was also carried out in duplicate as described above for Multiferon™ at a viral titre of 54 pfu/well.

Interferons are known to be relatively species specific as the target for the interferon is the infected cell rather than the virus itself. The anti-viral activity of Multiferon™ was also assessed in a human cell line, the pulmonary epithelial cell line A549.

Results

Cytopathic Endpoint Assay

The cytopathic effect of SARS-HCoV was evident within 24 hours following infection. Infected cells were rounded and exhibited monolayer destruction.

Complete inhibition using a high viral challenge (10⁴ pfu/well) and high multiplicity of infection (0.5) was observed for Ribavirin™, and for the Multiferon™ product. At a viral load of 10² pfu/well the CIA₁₀₀ value was 5 IU/ml for Multiferon™, with no cytotoxicity observed.

Although Ribavirin™ showed inhibitory activity at all viral titres this was only at high concentrations of the drug. Such concentrations showed cytotoxicity and thus Ribavirin™ is not likely to be a clinically effective treatment for severe acute respiratory syndrome (SARS) coronavirus.

In contrast, Multiferon™ did not show any cytotoxicity at this inhibitory concentration.

Interferon αn3, interferon αn1 and interferon β1b also showed inhibition of cytopathic effect using this assay. Interferon α2a, interferon α2b and interferon β1a did not show significant inhibition (results not shown).

Results are shown for Multiferon™ and Ribavirin™ in Tables 1 and 2 below.

TABLE 1
Results of the Cytopathic Endpoint Assay
for Multiferon ™ and Ribavirin ™. (Results not shown
for other treatments tested)

| | Concentration at | |
|---|---|---|
| | which complete | |
| Anti-viral | cytopathic | |
| Treatment | effect | CIA₁₀₀ |
| Multiferon ™ | 5,000 IU/ml | Yes |
| Ribavirin ™ | 5,000 μg/ml | Yes |

TABLE 2
Data obtained for Multiferon ™ and the anti-
viral product, Ribavirin ™. (Results not shown for
the other treatments tested).

| Virus Load | Multiferon ™ | Ribavirin ™ |
|---|---|---|
| (pfu/well) | (IU/ml) | (μg/ml) |
| 1,000 | 50 | 5,000 |
| 100 | 5 | 500 |

Plaque Reduction Assay

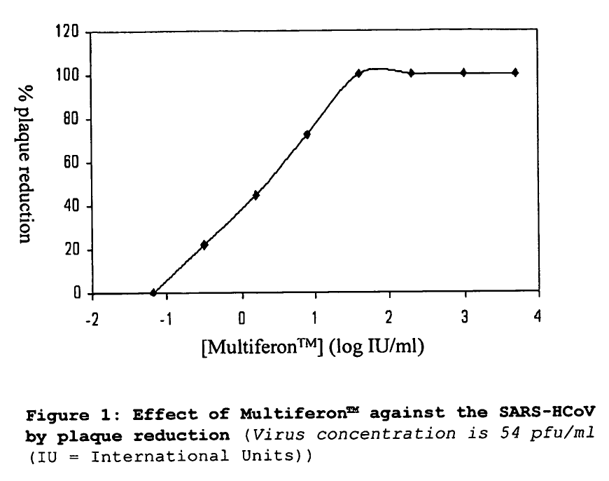

The Multiferon™ preparation displayed a dose-dependent inhibition of SARS-HCoV plaque formation. IC₅₀ and IC₉₅ values for Multiferon treatment were 2 IU/ml and 44 IU/ml, respectively. Results are shown below for Multiferon™ in Table 3 and in FIG. 1 for a viral titre of 54 pfu/well. An EC₅₀ value of 3.16 IU/ml was obtained.

TABLE 3
Results obtained in the plaque reduction
assay for Multiferon ™ at 54 pfu/well.

| Multiferon ™ | Log Multiferon ™ | % plaque | % plaque | Average |
|---|---|---|---|---|
| Concentration | Concentration | reduction | reduction | plaque |
| (IU/ml) | (Log IU/ml) | (Well 1) | (Well 2) | reduction |
| 5000 | 3.69897 | 100 | 100 | 100 |
| 1000 | 3 | 100 | 100 | 100 |
| 200 | 2.30103 | 100 | 100 | 100 |
| 40 | 1.60206 | 100 | 100 | 100 |
| 8 | 0.90309 | 68.5 | 75.9 | 72.2 |
| 1.6 | 0.20412 | 40.7 | 48.1 | 44.4 |
| 0.32 | −0.49485 | 18.5 | 25.9 | 22.2 |
| 0.064 | −1.19382 | 0 | 0 | 0 |

Interferon αn3 and interferon β1a also showed dose-dependent inhibition of SARS-HCoV plaque formation in this assay (results not shown).

### Example 2

SARS-HCoV, strain Frankfurt-1, kindly provided by the Bernard Notch Institute, Frankfurt, Germany, was propagated on Vero E6 cells, an African Green Monkey cell line obtained from American Type Culture Collection, Manassas, Va., USA. For titration of the virus, serial dilution of SARS-HCoV were added to Vero E6 cells grown in micro-plates with Eagle's medium containing 2% foetal calf serum. After 3 days of culture, cytopathogenic effects were determined microscopically and cytotoxity was then assayed using a calorimetric assay based on the measurement of lactate dehydrogenase (LDH) activity released from the cytosol of damaged cells (Cytotoxicity detection kit, Roche Diagnostics GmbH, Penzberg, Germany).

For the antiviral experiments the following four different commercially available interferon preparations were used: 1) Intron A™, Schering Plough, USA; 2) Roferon™, Roche, Switzerland; 3) Betaferon™, Schering AG, Germany and 4) Multiferon™ (Viragen, Fla., USA).

Serial 5-fold dilutions (0.2-31.125 IU/ml) of the interferon preparations were added to Vero E6 cells in micro-plates which were then incubated overnight at 37° C. SARS-HCoV was then added at different concentrations. (1000, 100 or 10 TCID₅₀) to different sets of interferon dilutions, and after a further incubation of 3 days the plates were read microscopically, and then by the ELISA LDH cytotoxicity assay.

In a separate set of experiments, the method used by Cinatl et al. (2003) including addition of interferon on two occasions, one day before and one day after addition of the virus to the plates, was employed.

In all experiments, controls with 1) virus but not interferon, 2) all different dilutions of the interferons but no virus, and 3) no virus and no interferon were included.

Results

The cytotoxicity (LDH) assay used for determination of SARS-HCoV cytopathogenic effect (CPE) was found to be highly reliable, giving OD values in CPE-positive cultures of 1.5-1.8 and in CPE-negative cultures values not exceeding 0.2.

Although two of the interferons, Roferon A™ and Multiferon™ showed a tendency to increase baseline levels in the cytotoxicity assay, the result showed no dose-dependent increase in these levels and the OD values did not exceed 0.6 in any case. There was no similar tendency for Intron A™ or Betaferon™ The concentration of interferons capable of decreasing OD values of virus-infected cultures by 50% (IC₅₀) are shown in Table 4 which shows the results of experiments where IFN was added either once (type 1) or twice (type 2) to the cells.

TABLE 4
Effect of various interferons on SARS-HCoV
replication

| | IC₅₀ Exp. Type 1 | | IL₅₀ Exp. Type 2 | |
|---|---|---|---|---|
| Interferon | 10 TCID₅₀ | 100 TCID₅₀ | 10 TCID₅₀ | 100 TCID₅₀ |
| Betaferon | 110 | 625 | 110 | 190 |
| Multiferon | 540 | 2400 | 490 | 2200 |
| Intron A | >3.125 | >3.125 | >3.125 | >3.125 |
| Roferon | >3.125 | >3.125 | >3.125 | >3.125 |

IC₅₀ values given as IU of interferon per ml. Slight inhibition of cytotoxicity was obtained with Roferon™ as well as Intron A™ at the highest concentrations tested, but the reduction of OD values did not reach the 50% level in any experiment with these interferons.

The outcome of the two different experiments performed were similar, showing that Betaferon™ had the highest antiviral activity (IC₅₀ 50-500 IU/ml) followed by Multiferon™ (IC₅₀ 500-2000 IU/ml). Neither Intron A™ nor Roferon™ had any clear antiviral activity at the highest concentrations used in the experiments (3.125 IU/ml). Extrapolation of results obtained with the highest concentrations of the IFN preparations showed that IC₅₀ levels could be expected to be reached at concentrations of 10,000-15,000 IU/ml for the latter two types of IFN-α.

Discussion

The present results corroborate earlier findings that IFN-β has an antiviral activity against the SARS-HCoV, that is superior to that of recombinant, IFN-β2, interferons (Cinatl et al., 2003). Furthermore, the results indicate that multi-subtype, natural IFN-α, albeit being less active that β-interferon, also has a significant effect on SARS-HCoV replication. The latter finding agrees with the recent results by Tan et al. (2004) who found, using a plaque reduction assay, that two types of natural IFN-α preparations showed strong anti SARS-HCoV activity with a potency that was only slightly lower than that obtained with β-interferon.

The accumulated evidence now suggests that interferons may have a role in the treatment of severe acute respiratory syndrome (SARS) coronavirus. The promising results of Loutfy et al. (2003) were obtained using a recombinant so-called consensus IFN-α (Infergen) that is believed to have effects that are shared by various subtypes of IFN-α. The suggestive clinically beneficial effect of the consensus IFN-α may be concordant with the presently obtained in vitro results with nIFN-α, but as far as we are aware, no studies on the relative in vitro activities of nIFN-α and consensus IFN-α have been performed.

### Example 3

### Anti-Viral Effect of Multi-Subtype Interferon as Compared to Intron A Against Semliki Forest Virus in Vero E6 Cells

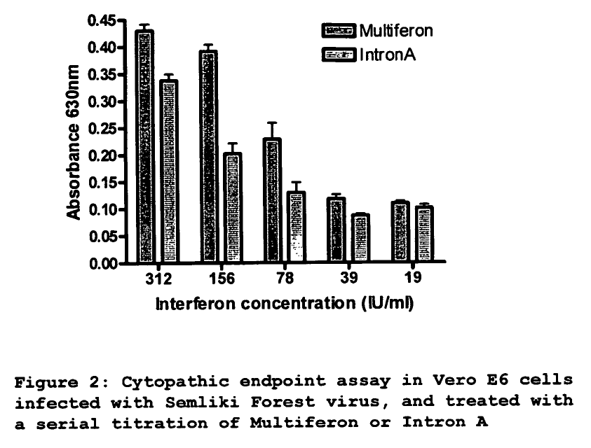

Vero E6 cells were seeded in 96-well plate, at a density of 10000 cells per well. After incubation overnight at 37° C., cells were incubated with 100 ul of a serial 10-fold dilution of Multiferon or Intron A (titration range from 1250 IU/ml-2.4 IU/ml). After 24 hours, cells were infected with 5000 pfu of Semliki Forest Virus (estimated MOI was 0.1) and further incubated for 48 hours until cytopathic effect was observed in untreated wells. Media was removed from cells, and cells were washed in 1× PBS, then fixed for 10 minutes at room temperature in 4% paraformaldehyde in PBS. Paraformaldehyde was removed and cells were stained with 0.2% crystal violet in 2% ethanol for 10 minutes at room temperature. Stained plates were washed and degree of colouration was quantified at 630 nm using an ELISA reader. Triplicate data is presented in graph format (FIG. 2).

Results

FIG. 2 demonstrates that Multiferon was found to be effective at protecting Vero E6 cells from SFV infection over a range of concentrations. At 625 IU/ml, the same degree of protection was observed for both Multiferon and IntronA (results not shown), and an equivalent loss of protection was observed for both products at 39 IU/ml. At all concentrations in between, Multiferon provided significantly higher protection that provided by Intron A.

### Example 4

### Anti-Viral Effect of Multi-Subtype Interferon in Human Cells

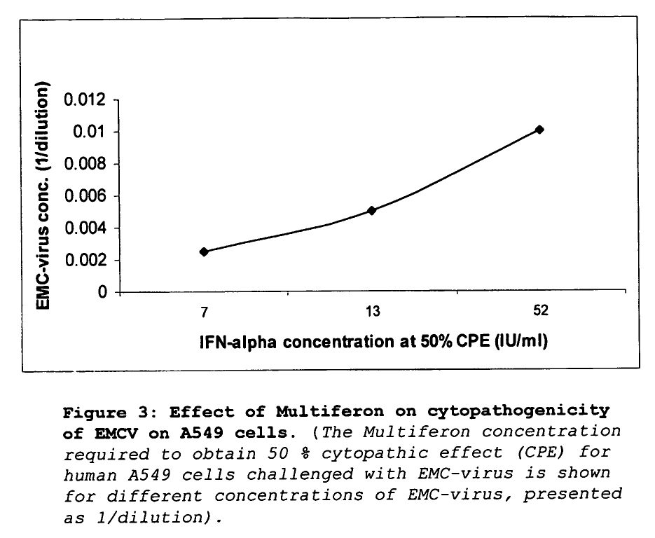

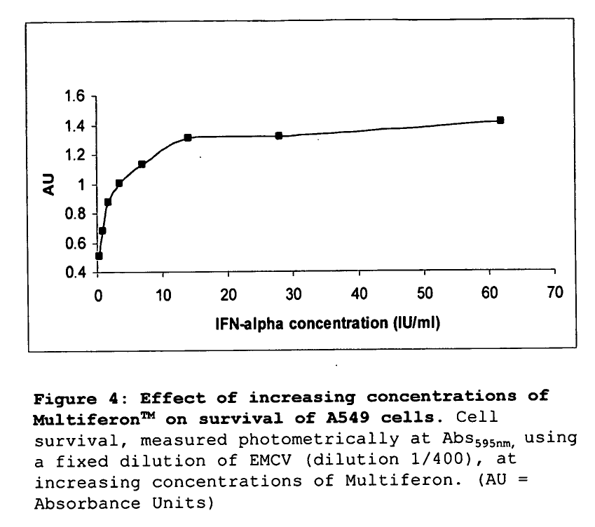

Multiferon™ was added prior to addition of the virus. The human Encephalomyocarditis virus (EMCV) was then used to infect A549 cells and the effect of Multiferon™ on the cytopathogenicity of EMCV was determined by assessing the interferon concentration required to obtain 50% cytopathic effect (CPE) for the human A549 cells. Results are shown in FIG. 3. Cell survival was measured photometrically and results are shown in FIG. 4.

The results show that the Multiferon™ preparation successfully protected against a cytopathic effect on EMCV-infected cells and that the adverse effect on the host cells did not continue to rise significantly at effective Multiferon™ concentrations.

FIG. 3 shows the concentrations of Multiferon™ needed to obtain 50% cytopathic effect in the human cells at varying viral titres. As would be expected, a higher viral concentration requires a higher effective Multiferon™ concentration.

FIG. 4 shows that Multiferon™ does not have significant adverse cell toxicity effects on human host cells.

Discussion

The results provided show that many interferons are highly effective at inhibiting the activity of the SARS-HCoV. Further, it has been shown that, in general natural interferons, especially multi sub-type interferons, such as Multiferon™, are particularly effective. Moreover at effective Multiferon™ concentrations, no cytotoxicity is observed.

In tests for anti-viral activity in human cells, Multiferon™ shows a good dose response with cytotoxicity levels which do not rise in proportion to the effective Multiferon™ concentration.

These results indicate that certain interferons such as Multiferon™ are highly effective therapeutics for the treatment of SARS-HCoV infection in humans and can be expected to have low levels of adverse effects in vivo.

Other groups have studied the efficacy of recombinant interferon products against SARS CoV. Stoher et al demonstrated significant but incomplete activity of Intron A at a concentration of 1000-5000 IU/ml on cells infected with a multiplicity of infection (MOI) of 0.001 plaque forming units per cell in a cytopathic endpoint assay. However, the results presented show that Multiferon™ used at the low dose of 5 IU/ml completely protected cells from SARS-HCoV infection at a MOI of 0.005 plaque forming units per cell, five times greater than the MOI used in the Intron A™ experiments. Furthermore, 50 IU/ml of Multiferon™ protected cells from SARS-HCoV infection at a MOI of 0.05, 50 times greater than the MOI utilised in the Intron A™ studies. Finally, in our studies, concentrations of Intron A™ or Roferon™ up to 100000 and 500000 IU/ml, respectively, failed to fully protect cells from SARS-HCoV infection.

Whilst Stoher et al. claim that doses of up to 3.6×10⁷ IU/ml have been infused intravenously, and that serum concentrations of at least 500 IU/ml are achievable after intramuscular injection, the serum titre would only reach this level for a short period of time, and intravenous infusion has highly toxic implications. Taken together with the results described, this supports the significant superiority of natural multi-subtype interferon products, in particular Multiferon™, over recombinant IFN alpha2 preparations.

All publications and patent documents referred to herein are incorporated by reference in their entirety. Although the invention has been described in connection with specific examples, it should be understood that the invention should not be unduly limited to such examples. Specifically, it will be understood by one skilled in the art that various modifications to and variations of the invention as described herein may be made without departing from the scope of the invention.

### REFERENCES

Al-Jabri et al. In Mahy, B W J and Kangro, H O eds. Virology Methods Manual, Academic Press Ltd, London (1996). 293-356
Cinatl, J et al. Lancet 362 (9380) 293-294
Crotty, S. et al. Nat. Med 6 1375-1379
Goodbourn, S. E. Y., et al. (2000). J. Gen. Virol. 81 2341-2364.
Lee, N. et al. N. Engl. J. Med. 348(20) 1986-1994
Loutfy, M. R. et al., JAMA 290(24) 3251-3253
Poutanen, S. M. et al. (2003) New Engl. J. Med. 348 (20) 1995-2005
Sidwell, R. W. et al. Antimicrob. Agents Chemother. 31 1130-1134
Stoher, U. et al. (2004). Journal of Infectious Diseases. 189:1164-7
Tam, R. C. et al. Antivir. Chem. Chemother. 12 (5) 261-272
Tan, E. L. C. et al. (2004). Emerging infectious diseases. 10(4) 581-586
Weck, P. K. et al. 1981. J. Gen. Virol. 57 233-237
Weiss, R. C. & Oostrom-Ram, T. Vet Microbiol. 20 255-265

1. A method of treating or preventing severe acute respiratory syndrome (SARS) coronavirus (SARS-HCoV) infection, the method including the step of administering a therapeutically useful amount of an interferon to a subject in need of treatment, wherein the interferon is a multi-type interferon consisting of interferon alpha (IFNα), interferon αn1, interferon αn3 or interferon β1b.

2. A method as claimed in claim 1 wherein the interferon is derived from human cells.

3. A method as claimed in claim 1 wherein the interferon is recombinant.

4. A method as claimed in any claim 1 wherein the interferon is an isolated interferon.

5. A method as claimed in claim 1 wherein the interferon is multi-subtype, human alpha-interferon derived from white blood cells commercially available as Multiferon™.

6. A method as claimed in claim 1 wherein the subject is human.

7. (canceled)

8. (canceled)

9. (canceled)

10. (canceled)

11. (canceled)

12. A method of treating infection with severe acute respiratory system (SARS) coronavirus (SARS-HCoV), the method including the step of administering a therapeutically useful amount of an interferon to a subject in need of treatment along with a therapeutically useful amount of a suitable anti-viral compound, wherein the interferon is a multi-subtype interferon consisting of interferon alpha (IFNα), interferon αn1, interferon αn3 or interferon β1b.

13. A method as claimed in claim 12 wherein the interferon is the multi-subtype, human alpha-interferon derived from white blood cells commercially available as Multiferon™.

14. A method as claimed in claim 12 wherein the anti-viral compound is ribavirin.

15. A method as claimed in claim 12 wherein the subject is human.

16. A combined medicament comprising an interferon and an anti-viral compound for use in the treatment or prevention of infection with a severe acute respiratory system (SARS) coronavirus (SARS-HCoV), wherein the interferon is a multi-subtype interferon consisting of interferon alpha (IFNα), interferon αn1, interferon αn3 or interferon β1b.

17. A combined medicament as claimed in claim 16 wherein the interferon is the multi-subtype, human alpha-interferon derived from white blood cells commercially available as Multiferon™.

18. A combined medicament as claimed in claim 16 wherein the infection is infection of a human.

19. An assay method for determining the efficacy of a candidate agent in the treatment of infection with a severe acute respiratory system (SARS) coronavirus (SARS-HCoV), the assay method including the steps of:
incubating cells infected with coronavirus in the presence of the candidate agent,
determining the degree of inhibition of the cytopathic effect of the virus on the cells, and
comparing the degree of inhibition obtained using the candidate agent with the degree of inhibition obtainable with incubation with a multi-subtype interferon consisting of interferon alpha (IFNα), interferon αn1, interferon αn3 or interferon β1b, or a product based on these inteferons.

20. An assay as claimed in claim 19 wherein the interferon is the commercially available multi-subtype interferon alpha composition Multiferon™.

21. An assay as claimed in claim 19 wherein the infection is infection of a human.

22. (canceled)

23. (canceled)

24. (canceled)

25. A multi-subtype interferon consisting of interferon alpha (IFNα), interferon αn1, interferon αn3 or interferon β1b for use in the treatment or prophylaxis of a severe acute respiratory syndrome (SARS) coronavirus (SARS-HCoV) infection.

26. A multi-subtype interferon as claimed in claim 7 wherein the interferon is human alpha-interferon derived from white blood cells commercially available as Multiferon™.

27. A multi-subtype interferon as claimed in claim 7 wherein the interferon is recombinant interferon.
